(19) United States
(12) Patent Application Publication
(10) Pub. No.: US 2021/0177669 A1
(43) Pub. Date: Jun. 17, 2021

(54) FLEXIBLE AND COMPLIANT MECHANICALLY-DEFORMED NONWOVENS FOR USE IN ABSORBENT ARTICLES

(71) Applicant: The Procter & Gamble Company, Cincinnati, OH (US)

(72) Inventors: Andrea Peri, Kronberg (DE); Ernesto Gabriel BIANCHI, Schwalbach (DE); Sara Lyn GIOVANNI, Cincinnati, OH (US); Adrien GRENIER, Schwalbach (DE); Rolf HECKER, Schwalbach (DE); Matthias Konrad HIPPE, Schwalbach (DE); Timothy Ian MULLANE, Union, KY (US); Mattias SCHMIDT, Schwalbach Am Taunus (DE)

(21) Appl. No.: 17/113,333

(22) Filed: Dec. 7, 2020

Related U.S. Application Data
(60) Provisional application No. 62/946,562, filed on Dec. 11, 2019.

(51) Int. Cl. A61F 13/514; A61F 13/511

(52) U.S. Cl. CPC A61F 13/51476; A61F 13/51478; A61F 2013/51494; A61F 13/51108; A61F 2013/51452; A61F 13/5116

(57) ABSTRACT

A mechanically deformed nonwoven comprising a plurality of protrusions extending outwardly from a first surface of the nonwoven and having openings c corresponding to the protrusions in a second surface of the nonwoven. The nonwoven has a Horizontal Bending Drop at 100 mm of at least 75 mm and a Z-Compliance Index of at least 50 mm³/N. The nonwoven can be used in absorbent articles such as diapers as an acquisition layer between the absorbent core and the backsheet and/or as a masking layer between an absorbent layer core and the backsheet.

### CROSS REFERENCE TO RELATED APPLICATION

This application claims the benefit of U.S. Provisional Application No. 62/946,562, filed on Dec. 11, 2019, the entire disclosure of which is fully incorporated herein by reference.

### FIELD OF THE INVENTION

The present disclosure generally relates to nonwovens that can be used in disposable absorbent articles for personal hygiene such as diapers, feminine care pads or adult incontinence products. The nonwovens may be for example used as an acquisition layer between the topsheet and the absorbent core of the article, and/or as a masking layer between the absorbent layer and the backsheet.

### BACKGROUND OF THE INVENTION

Disposable absorbent articles such as feminine hygiene products, taped diapers, pant-type diapers and incontinence products are designed to absorb and contain body fluids from the wearer's body. There is a continuous need to provide absorbent articles that have good absorbency, feel soft to touch, and are economical to produce.

Various constructions have been proposed for absorbent articles. Most absorbent articles comprise today the following layers, as considered from the wearer-facing side to the garment facing-side: a liquid permeable topsheet, an acquisition layer and/or a distribution layer, an absorbent core and a liquid impermeable backsheet. The absorbent core comprises superabsorbent polymers (SAP), typically in particulate form, that can absorb many times their own weight of urine. The SAP may be mixed with cellulose fibers to form a mixed absorbent layer having good fluid acquisition and retention capabilities. However, these mixed layers may be relatively bulky due to the cellulose fibers and the void volume between them. More recently, absorbent cores that are substantially free of cellulose fibers have been commercialized. US2008/0312617 and US2010/0051166A1 (both to Hundorf et al., P&G) for example disclose absorbent articles having substantially cellulose free absorbent cores comprising one or two layers of SAP particles which are immobilized by a thermoplastic adhesive material in a core wrap. A distribution layer comprising cross-linked cellulose fibers is disclosed for use with these absorbent cores.

While these absorbent articles are thin and provide good absorbency, the adhesive-immobilized SAP particles can cause a grainy texture at the surface of the diaper, especially at the back of the article that is not covered by the acquisition/distribution system. Also, the cross-linked cellulose fibers used as to form the distribution layer of the diaper can become clumpy during use, which provides a poor appearance to the diaper.

It is desirable to improve the haptic perception of absorbent articles. For this, the inventors have found that multiple factors that need to be satisfied at the same time. For example, a diaper needs to have enough cushiness when compressed so that it provides padding to the delicate genital area, but at the same time it needs to be flexible to wrap around the wearer during motion like a textile. These properties are however in general in contradiction. In other words when cushiness is increased (for example by increasing the basis weight and/or overall amount of the soft material used), the flexibility is often compromised.

There is therefore a need to find materials that can be used in absorbent articles, and which are at the same time cushy and highly flexible, while being economical to produce for disposable absorbent articles.

### SUMMARY OF THE INVENTION

The invention is, in a first aspect, for a mechanically deformed nonwoven comprising a plurality of protrusions extending outwardly from a first surface of the nonwoven and having openings corresponding to the protrusions on the second surface of the nonwoven. This deformation provides the nonwoven with a Horizontal Bending Drop at 100 mm of at least 75 mm, and a Z-Compliance Index of at least 50 mm³/N. The Horizontal Bending Drop and the Z-Compliance Index characterize the flexibility and cushiness of the nonwoven respectively. The measurement methods are described further below. Further advantageous features are as indicated in the dependent claims and further described in this description.

In a second aspect, the invention is for an absorbent article, in particular a diaper in taped or pant-type form, comprising such a nonwoven according to the first aspect of the invention. The nonwoven may in particular be used between the topsheet and the absorbent core as an acquisition layer. Such an acquisition layer can deliver the required perception of softness while maintaining an adequate level of acquisition performance. The absorbent core may comprise at least one absorbent layer comprising superabsorbent particles, which may be free of cellulose fibers. The nonwoven may alternatively or in combination be used as a masking layer between the absorbent layer and the backsheet, either as integral part of the absorbent core or between the absorbent core and the backsheet. Such masking layer can effectively from a screen between the layer of superabsorbent particles and the backsheet, and thus prevent the graininess of the superabsorbent particles to be felt through the backsheet.

### BRIEF DESCRIPTION OF THE DRAWINGS

FIG. 1 shows a perspective view of an exemplary taped diaper in a closed configuration as it would be when worn by a wearer (not represented);

FIG. 2 shows a simplified view of the garment-facing side of the diaper of FIG. 1 with the diaper flattened out;

FIG. 3 shows a simplified view of the wearer-facing side of the diaper of FIG. 1 with the diaper flattened out;

FIG. 4 shows a first possible schematic cross-section of the diaper of FIGS. 1-3;

FIG. 5 shows a schematic close up view of an exemplary protrusion having a bulbous shape;

FIG. 6 shows a schematic close up view of an exemplary protrusion having a bulbous shape as in FIG. 5 but where a dual layer nonwoven has been deformed;

FIG. 7 shows a schematic close up view of an exemplary protrusion having a bulbous shape and a secondary opening at its cap;

FIG. 8 shows a protrusion similar to as in FIG. 7 but where a dual layer nonwoven has been deformed;

FIG. 9 shows an alternative deformed nonwoven where protrusions have been formed on both sides of the nonwoven;

FIG. 10 shows protrusions similar to FIG. 9 but where the nonwoven is a dual layer nonwoven; and

FIG. 11 shows a schematic setup for conducting the Horizontal Bending Drop Test.

### DETAILED DESCRIPTION OF THE INVENTION

### Definitions

The term “nonwoven” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be hydrophilic or hydrophobic as desired, based on the inherent properties of the fibers or via treatment e.g. addition of a surfactant on the fibers.

“Absorbent article” refers to wearable devices, which absorb and/or contain liquid, and more specifically, refers to devices, which are placed against or in proximity to the body of the wearer to absorb and contain the various exudates discharged from the body. Absorbent articles considered include baby diapers, training pants, adult incontinence undergarments (e.g., liners, pads and briefs) and/or feminine hygiene products.

“Machine Direction” or “MD” as used herein means the direction parallel to the flow of the nonwoven through the nonwoven making machine and/or absorbent article manufacturing equipment.

“Cross-Machine Direction” or “CD” as used herein means the direction parallel to the width of the nonwoven making machine and/or absorbent article manufacturing equipment and perpendicular to the machine direction.

The “Z-direction” is orthogonal to both the Machine Direction and the Cross-Machine Direction.

### General Description of a Mechanically Deformed Nonwoven 60 According to the Invention

The nonwovens of the invention can be produced from a wide variety of nonwoven precursor, exemplified further below, which are mechanically deformed so that some of the fibers of the nonwoven precursor are pushed out of the plane of the precursor nonwoven to form hollow protrusions extending from at least one surface of the nonwoven, with corresponding openings on the other surface. Examples of mechanical processes to deform the precursor nonwovens are also further disclosed further below.

The inventors have found that by mechanically deforming such two-dimensional precursor nonwovens, three-dimensional nonwovens are obtained having enhanced conformability and compliance. While some selected material nonwovens such as spunlace may have already good properties without a mechanical treatment, it was found that mechanically deforming many types of nonwovens can generally improve the desirable properties of the nonwoven. These mechanically deformed nonwoven can be used in absorbent articles, e.g. as an acquisition layer or as part of an acquisition-distribution system and/or as a masking layer.

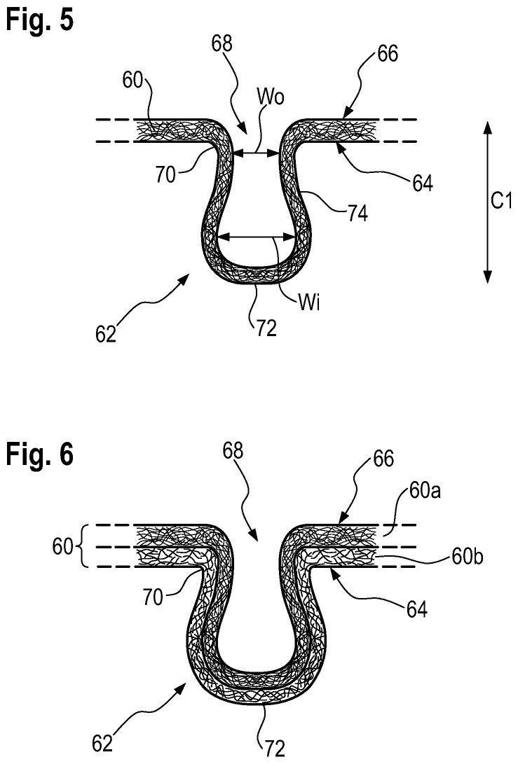

A close-up schematic drawing of an exemplary protrusion 62 according to the invention is illustrated on FIG. 5. The reference number 60 in the drawings is pointing to a part of the nonwoven which has not been deformed, and thus is representative of the precursor nonwoven. The precursor nonwoven may be a single material as illustrated in FIG. 5, or a composite nonwoven comprising two or more sub-layers 60a, 60b as illustrated in FIG. 6. The protrusions 62 extend from a first surface 64 of the nonwoven 60 and have on the opposite second surface 66 a corresponding opening 68.

The mechanically deformed nonwovens of the present invention have a Horizontal Bending Drop at 100 mm (HBD@100 mm) of at least 75 mm and a Z-Compliance Index of at least 50 mm³/N. These properties are measured as indicated in the measurement method described further below. The HBD@100 mm may be advantageously at least 80 mm, and even more advantageously at least 85 mm. The maximum theoretical value for the HBD@100 mm is 100 mm, but as indicated in the background having increased flexibility is difficult to combine with cushiness. The HBD@100 mm may thus typically be up to 99 mm, or up to 98 mm or even up to 96 mm. A mechanically deformed nonwoven according to the present invention may thus have a HBD@100 mm ranging of from 75 mm up to 99 mm, or from 80 mm up to 98 mm, or from 80 mm up to 97 mm.

The Z-Compliance Index measures the cushiness of the nonwoven. The mechanically deformed nonwoven of the invention has a Z-Compliance Index of at least 50 mm³/N. Advantageously, the Z-Compliance Index of the nonwoven may be of at least 55 mm³/N, or at least 60 mm³/N, or at least 65 mm³/N, or at least 70 mm³/N. There is no theoretical maximum value for the Z-Compliance Index, but in practice in order to reach a good compromise between cushiness and flexibility, the Z-Compliance Index of the nonwoven according to the invention may be up to 150 mm³/N, or up to 120 mm³/N, or up to 100 mm³/N, or up to 90 mm³/N.

The deformed nonwovens of the invention may advantageously have a thickness and basis weight that also define a good compromise for the desired properties. A lower basis weight material and/or thinner material may be more economical and more bendable, but may have less cushiness than the same material at higher basis weight and/or thicker material. Thus, the deformed nonwoven of the invention may have a thickness (also named “caliper”) ranging from 0.50 mm to 4.00 mm, in particular of from 1.00 mm to 3.00 mm, as measured at a pressure of 0.85 kPa. This caliper is designated as C1 (FIG. 5) and can be measured as described in the Z-Compliance Index and % Recovery Measurement Method, described further below. The nonwoven may also have a basis weight ranging of from about 10 grams per square meter (gsm) to about 140 gsm, in particular from 20 gsm to 120 gsm, or from 30 gsm to 100 gsm. The caliper of the nonwoven is typically increased by the deformation process, while the basis weight may be typically somewhat decreased if the nonwoven is stretched and elongated during the deformation.

The mechanically deformed nonwovens of the invention may further have a Percent Recovery of at least 50%, or at least 60%, or at least 65%, or at least 70%, as measured by the Z-Compliance Index and Percent Recovery Measurement Method described herein. The theoretical maximum Percent Recovery value is 100%. In order retain cushiness over the product usage, suitable nonwovens may have Percent Recovery of up to 95%, or up to 90% or even up to 85%. Suitable Percent Recovery for the nonwoven of the invention may thus be in the range of from 50% up to 95%, or from 60% to 90%, of from 65% up to 85%.

### Precursor Nonwovens

A wide variety of precursor nonwovens may be mechanically deformed to obtain the required properties in the deformed nonwoven. The nonwoven precursor can be made from a single layer, or multiple layers (e.g. two or more layers). The term “precursor nonwoven” as used herein refers to any type of nonwovens (single layer, composite layer and integrated layer) that has been deformed to form the deformed nonwoven of the invention. If the precursor nonwoven comprises multiple sub- or integrated layers, these can comprise the same type of fibers or have different fiber composition. In some cases, the precursor materials may be free of any film layers. The precursor nonwovens are also typically free of superabsorbent polymer particles (SAP).

The fibers of the nonwoven precursor(s) can be made of any suitable materials including, but not limited to, natural materials, synthetic materials, and combinations thereof. Suitable natural materials include, but are not limited to cellulose, cotton linters, bagasse, wool fibers, silk fibers, regenerated cellulose such as viscose or rayon etc. Cellulose fibers can be provided in any suitable form, including but not limited to individual fibers, fluff pulp, drylap, liner board, etc. . . . . Such fibers may be inherently hydrophilic. Suitable synthetic polymeric materials include, but are not limited to, polyethylene (PE), polyester, polyethylene terephthalate (PET), polypropylene (PP), and co-polyester. One or more precursor materials can comprise up to 100% thermoplastic fibers and be hydrophobic, unless it has been treated to hydrophilic, e.g. by a surfactant as is known in the art. The fibers in some cases may, therefore, be substantially non-absorbent. The synthetic fibers may be monocomponent, bicomponent, and/or biconstituent, non-round (e.g. shaped fibers (including but not limited to fibers having a trilobal cross-section) and capillary channel fibers).

The fibers can be of any suitable size. The fibers may, for example, have major cross-sectional dimensions (e.g. diameter for round fibers) ranging from 0.1-500 microns. Fiber size can also be expressed in denier, which is a unit of weight per length of fiber. The constituent fibers may, for example, range from about 0.1 denier to about 100 denier. The constituent fibers of the nonwoven precursor(s) may also be a mixture of different fiber types, differing in such features as chemistry (e.g., PE and PP), components (mono- and bi-), shape (i.e. capillary channel and round) and the like.

The nonwoven precursors can be formed from many processes, such as for example, air laying processes, wetlaid processes, meltblowing processes, spunbonding processes, and carding processes. The fibers in the precursor webs can then be bonded via any known processes such as spunlacing processes, hydroentangling, calendar bonding, through-air bonding and latex (resin) bonding. Some of such individual nonwoven precursor webs may have bond sites where the fibers are bonded together.

The basis weight of nonwoven materials is usually expressed in grams per square meter (gsm). The basis weight of the precursor nonwoven material (whether single layer, composite layer or integrated layer) can typically range from about 10 gsm to about 140 gsm, in particular from 20 gsm to 120 gsm, or more particularly of from 30 gsm to 100 gsm, depending on the ultimate use and targeted cost of the material. The basis weight of a multi-layer material is the combined basis weight of the constituent layers and any other added components. The nonwoven precursors typically have a density ranging from about 0.05 g/cm³ to about 0.4 g/cm³, more particularly from about 0.1 g/cm³ to about 0.3 g/cm³, measured at 0.85±0.05 kPa.

The precursor nonwoven each have a first surface 64, a second surface 66, and a thickness. The first and second surfaces of the precursor nonwovens may be generally planar before the deformation. It is typically desirable for the precursor nonwovens to have extensibility to enable the fibers to stretch and/or rearrange into the form of the protrusions. If the precursor nonwovens are comprised of two or more layers, it may be desirable for all of the layers to be as extensible as possible. Extensibility is desirable in order to maintain at least some non-broken fibers in the sidewalls around the perimeter of the protrusions. It is also desirable for the precursor nonwoven webs to be capable of undergoing plastic deformation to ensure that the structure of the deformations is “set” in place so that the nonwoven web will not tend to recover or return to its prior configuration.

A few example of nonwoven types that can be deformed are discussed below, in a non-limiting way.

Airlaids:

The precursor nonwoven may be an airlaid nonwoven, comprising short fibers that are either 100% cellulose pulp fibers, or preferably mixtures of pulp fibers and short cut synthetic fibers, to form a homogeneous and continuous web. As known in the art, airlaid can be bonded in several ways, in particular via latex bonding (LBAL), thermal bonding (TBAL) and multi bonding (MBAL). In latex bonding, a liquid binder is applied to either or both sides of the web, which is thereafter dried and cured to achieve the dry and wet strength needed. In thermal bonding, bonding fibers such bicomponent fibers, are included in the web formation, and the web is heated to activate the melting components of the synthetic fibers to bond the web. Multi bonding is a bonding process where latex and thermal bonding are combined, typically where the inner part of the product is thermal bonded and the surfaces have a slight layer of binder to eliminate dust and linting.

Composite Nonwovens:

The precursor nonwoven may be a composite nonwoven comprised of two or more layers, which are in close contact and deformed simultaneously, as illustrated in FIG. 6. Examples of such precursor composite nonwovens and their method of fabrication are described for example in WO2018/197937 (Ren et al., Fitesa). The composite nonwoven may in particular comprise:
a fluid acquisition component 60a, in particular comprising a carded nonwoven fabric; and
an airlaid component 60b in face to face contact with the fluid acquisition component 60a, the airlaid component in particular comprising a blend of cellulose staple fibers and non-cellulose staple fibers.

The fluid acquisition component 60a and the airlaid component 60b may be thermally bonded at their interface, with their outer surface respectively forming the first surface 64 and second surface 66 of the composite nonwoven sheet 60.

A method for making such a composite nonwoven comprises the steps of providing a carded nonwoven fabric, the carded nonwoven fabric comprising staple fibers; and depositing an airlaid layer onto a surface of the carded nonwoven fabric (used as a carrier layer) to form a composite sheet, the airlaid layer comprising a mixture of cellulose and non-cellulose staple fibers; and bonding the composite sheet with heated gas to cause a polymer of the non-cellulose staple fibers to melt and fuse with adjacent fibers.

The airlaid component 60b may comprise cellulose fibers up to 90%, or from about 50% to about 85%, or from about 60% to about 80%, by weight of the airlaid component. A wide variety of polymers may be used for the thermoplastic fibers. Examples of suitable fibers include polyolefins such as polypropylene and polyethylene, and copolymers thereof, polyesters such as polyethylene terephthalate (PET), polytrimethyene terephthalate (PTT), and polybutylene terephthalate (PBT), nylons, polystyrenes, copolymer or blends thereof, and other synthetic polymers conventional in the preparation of fibers.

Suitable materials for the thermoplastic fibers include monocomponent or multicomponent fibers, or mixtures thereof. The thermoplastic fiber may comprise a sheath/core bicomponent fiber. The sheath/core bicomponent fiber may comprises a sheath comprising a polymer having a lower melting temperature than that of a polymer forming the core. The lower melting polymer of the sheath may promote bonding while the higher melting polymer of the core may provide strength to the thermoplastic fiber and thus to the first component. The thermoplastic fibers typically have lengths ranging from about 3-15 mm, or from about 3-10 mm, or from about 3-6 mm. In some embodiments, the sheath/core bicomponent fibers may comprises PE/PET fibers, PE/PP fibers or a mixture thereof. In the airlaid component, the thermoplastic fibers may be thermal bonded and may entrap cellulose fibers.

The airlaid component may further comprise a binder such as latex. The binder may help immobilizing the cellulose fibers. The airlaid component may have a basis weight in the range of about 20 gsm-140 gsm, or about 30 gsm-120 gsm, or about 40 gsm-80 gsm. A basis weight of the airlaid component may be determined to balance acquisition-distribution performance and a thickness of the absorbent article.

The fluid acquisition component 60a may comprise second thermoplastic fibers. Examples of second thermoplastic fibers include thermoplastic fibers discussed with respect to the first component. The second thermoplastic fibers may or may not be the same as the first thermoplastic fiber. The fluid acquisition component may be free of cellulose fibers. The fluid acquisition component may have a basis weight in the range of about 20-80 gsm, or about 30-70 gsm, or about 40-60 gsm.

The fluid acquisition component 60a may comprise or consists of a carded nonwoven, in particular an air-through bonded carded nonwoven. The fluid acquisition component may alternatively comprise or consist of a spunbond nonwoven or spunbond-meltblown-spunbond (“SMS”) nonwoven. SMS can mean a three layer, ‘sms’ nonwoven materials, a five layer ‘ssmms’ nonwoven materials, or any reasonable variation thereof wherein the lower case letters designate individual layers and the upper case letters designate the compilation of similar, adjacent layers.

The precursor composite nonwoven may also comprise one or more additional layers for example deposited on the outer surface of the airlaid component. More generally, any of the layers of the composite nonwoven may comprise or consists of a carded web, air-laid web, wet-laid web, and spunbond web, and the like.

Integrated Nonwovens:

A particular type of suitable precursor nonwovens are integrated nonwovens. Integrated nonwovens comprise strata of fibers which have been integrated at their interface. Fiber integration of a nonwoven can occur via any suitable process which entangles fibers primarily in a Z-direction (positive or negative). Exemplary processes which are amenable in creating such fiber integration include needlepunching and spunlacing. Needlepunching involves the mechanical interlocking of fibers of spunbonded and/or carded web(s). In the needlepunching process, a plurality of barbed needles repeatedly passes in and out of nonwoven web(s) and push fibers of the nonwoven web(s) in a positive and/or negative Z-direction. In contrast, the spunlace process uses high-speed jets of water to cause the interlocking of fibers of a nonwoven web(s). The high-speed jets of water push fibers of the nonwoven web in the positive or negative Z-direction. With the aid of a microscope, needlepunched nonwovens comprise a plurality of discrete Z-direction fiber integrations in both MD and CD directions, while spunlace nonwovens generally comprise much more continuous integrations along the MD direction but discrete in the CD direction.

The term “spunlace” means a nonwoven wherein the cohesion and the interlacing of the fibers with one another is obtained by means of a plurality of jets of water under pressure passing through a moving fleece or cloth and, like needles, causing the fibers to intermingle with one another. These spunlace nonwovens are essentially defined by the fact that their consolidation results from hydraulic interlacing.

“Spunlace”, as used herein, also relates to a nonwoven formed of two or more webs (stratum), which are combined with each other by hydraulic interlacing. The two webs, prior to being combined into one nonwoven by hydraulic interlacing, may have underdone bonding processes, such as heat and/or pressure bonding by using e.g. a patterned calendar roll and an anvil roll to impart a bonding pattern. However, the two webs are combined with each other solely by hydraulic interlacing.

The precursor nonwoven has a first surface 64 and an opposing second surface 66. The precursor nonwoven of the present invention may in particular comprise two, three or more strata along the Z-direction which have been integrated as described above between these two surfaces. These strata are typically carded webs made of staple fibers.

Due to the fiber integration, the integrated nonwoven may not require adhesives or latex binders for stability. Additionally, a carded staple fiber nonwoven can be manufactured from an assortment of suitable fiber types that produce the desired performance characteristics. In particular, the precursor nonwoven of the invention may comprise a combination of absorbent fibers, stiffening fibers and resilient fibers.

In order to enhance the stabilizing effect of the integration, one or more of these fibers may be crimped prior to integration. For example, where synthetic fibers are utilized, these fibers may be mechanically crimped via intermeshing teeth. And for the absorbent fibers, these fibers may be mechanically crimped and/or may have a chemically induced crimp due to the variable skin thickness formed during creation of the absorbent fibers.

Overall, spunlace nonwoven used in the invention may comprise from about 20 percent to about 75 percent by weight, or from about 25 percent to about 60 percent by weight, or from about 30 percent to about 50 percent by weight, specifically including any values within these ranges and any ranges created thereby, of absorbent fibers.

Overall, spunlace nonwoven of the invention may comprise (by weight) from about 1 percent to about 50 percent, or from about 10 percent to about 40 percent, or from about 20 percent to about 30 percent of stiffening fiber, specifically reciting all values within these ranges and any ranges created thereby.

Overall, the spunlace nonwoven of the present invention may comprise from about 10 percent to about 50 percent, or from about 13 percent to about 40 percent, or from about 20 percent to about 35 percent, or from about 25 percent to about 30 percent by weight of resilient fibers, specifically reciting all values within these ranges and any ranges created thereby.

### Mechanical Deformation

The nonwovens according to the invention are produced by mechanically deforming precursor nonwovens such as those disclosed above in order to improve their mechanical properties. Some precursor nonwovens, such as some integrated spunlace, may already have good properties in or close to the range desired, however it was found that mechanical deformation can even for these materials further increase their performance as measured by a Horizontal Bending Drop at 100 mm and the Z-Compliance Index, as discussed further below.

Generally, the deformed nonwovens are made by a method comprising the steps of: a) providing at least one precursor nonwoven; b) providing an apparatus comprising a pair of forming members comprising a first forming member (e.g. a “male” forming member) and a second forming member (e.g. a “female” forming member); and c) placing the precursor nonwoven(s) between the forming members and mechanically deforming the precursor nonwoven(s) with the forming members. The forming members have a machine direction (MD) orientation and a cross-machine direction (CD) orientation. The first and second forming members can be plates, rolls, belts, or any other suitable types of forming members. The mechanical deformation typically includes passing the precursor nonwoven web between two rolls having a specific intermeshing pattern on their surfaces at certain depth of engagement (DOE).

As a result of the mechanical deformation, a plurality of protrusions 62 extending outwardly from a first surface 64 are formed by displacing fibers of the nonwoven away from the first surface. Simultaneously, openings 68 corresponding to the protrusions are formed on the second surface 66 of the nonwoven. The plurality of protrusions thus formed are preferably discrete protrusions. The mechanical deformation process is different from a conventional embossing process where fibers are compressed inwardly and do not form outwardly extending protrusions.

Various apparatus and methods for making such three-dimensional protrusions have been disclosed in the art. U.S. Pat. No. 8,502,013 (Zhao et al., P&G) for example illustrates in FIG. 6 and following a pair of inter-meshing rolls for a process commonly referred to a SELF process, that may be used to deform a precursor nonwoven. FIG. 14 and following of this reference illustrate a rotary knife apparatus (RKA) and process that uses pointed, pyramidal-shaped teeth on one roll which may provide secondary opening at the tip of the protrusions.

WO2016/040101A1 (Strube et al., P&G) discloses a nonwoven deformation process (referred to as nested-SELF process) to make a nonwoven having discrete three-dimensional bulbous protrusions 62 with wide base openings 68, similar to those illustrated in FIG. 5-8. In this process, with an exemplary apparatus is shown on a perspective view FIG. 21 and following of WO2016/040101A1, the first and second forming members are in the form of non-deformable, meshing, counter-rotating rolls that form a nip therebetween. The precursor nonwoven is fed into the nip between the rolls. Although the space between the rolls is described herein as a nip, as discussed in greater detail in WO2016/040101A1, in some cases, it may be desirable to avoid compressing the precursor nonwovens(s) to the extent possible.

This nested-SELF process is advantageous as it provides bulbous protrusions 62 as shown on FIGS. 5-8. These protrusions comprise a base 70 proximate the first surface 64 of the nonwoven, an opposed distal end extending outward in the Z-direction from the base, side walls 74 between the base and the distal end of the protrusion 62, and a cap 72 comprising at least a portion of the side walls and the distal end of the protrusions. The side walls 74 have interior surfaces, wherein the interior surfaces of the side walls define a base opening 68 at the base of the protrusion, wherein the cap 72 has a portion with a maximum interior width Wi, and the base opening 68 has a width Wo, wherein the maximum interior width Wi of the cap 72 of the protrusion is greater than the width Wo of the base opening 68. These bulbous protrusions are particularly resilient and can e.g. typically at least partially recover their shape or at least not collapse under compression in the package of absorbent article.

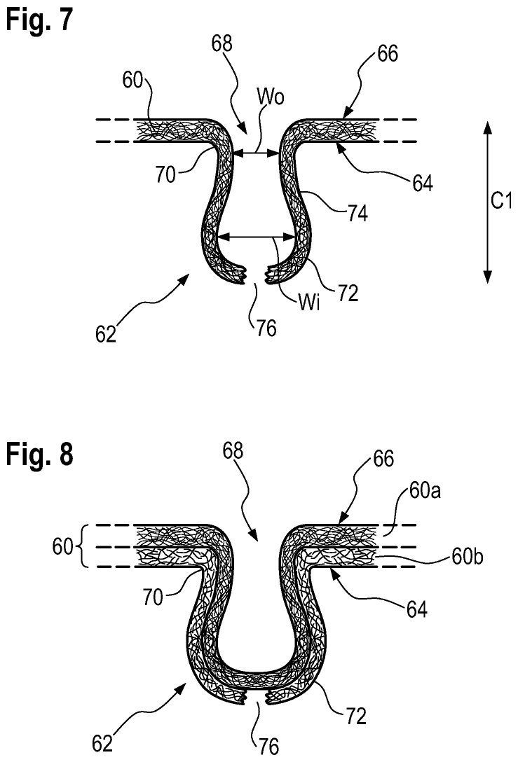

The processes may also be conducted such that one or more secondary openings 76 are formed at the cap 72 or the distal end of the protrusions, due to the fibers of the precursor nonwoven not being sufficiently elongatable and breaking at the tip of the protrusions as illustrated in FIG. 7. This may be desired when the nonwoven is used as acquisition layer so that a fluid can more quickly move through the nonwoven towards the absorbent core. When a multi-layer precursor nonwoven is deformed, the second opening 76 may extend through all the layers of the nonwoven, or only be formed in one layer, for example the one forming the external surface of the deformed nonwoven as represented on FIG. 8.

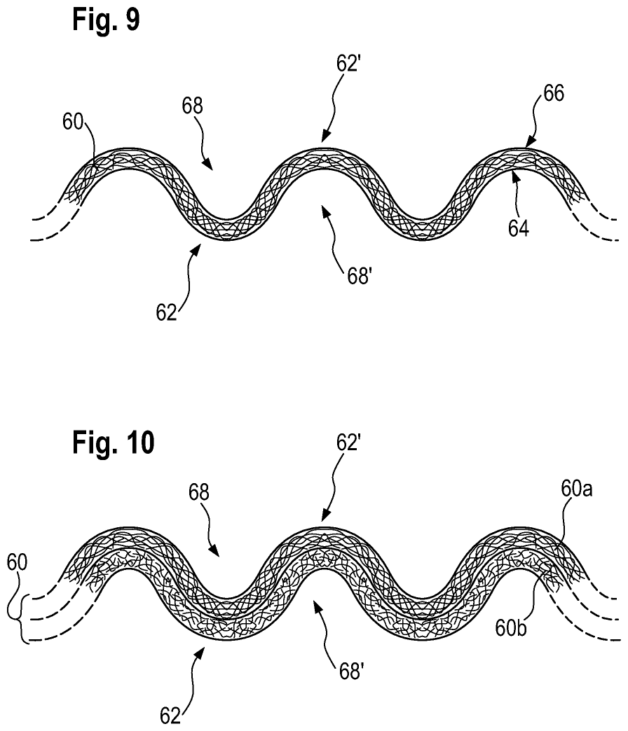

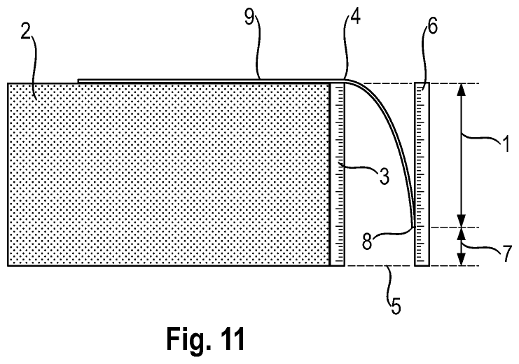

Another related mechanical deformation process that may be used to make deformed nonwoven according to the invention is disclosed in WO2012/148,944 (Marinelli et al., P&G), where two rolls having intermeshing male elements are used to deform the nonwoven web. This is process may be referred to as SELF-on-SELF (SoS) and is illustrated on FIG. 16 and following of WO2012/148,944A1. The deformed nonwoven obtained by such a process is schematically represented in FIGS. 9-10, where protrusions 62 are formed on a first surface of the nonwoven and equivalent protrusion 62′ are formed on the second surface of the nonwoven, with the corresponding openings 68, 68′. The SoS process can be used on a single layer nonwoven (as illustrated in FIG. 9) or dual or multi layer nonwoven (as illustrated in FIG. 10).

Generally, the protrusions may be uniformly distributed on the forming members and thus on the deformed nonwoven. The protrusions may also be distributed according to a pre-determined pattern by arranging the male and/or female elements on the forming elements, in particular forming rolls, according to a desired pattern. The average number of protrusions on the deformed nonwoven may typically range from 0.5 to 5 per square centimeter. The flexibility of the deformed nonwoven as measured by the HBD test will typically depend on which side of the deformed nonwoven is facing up, as the protrusions may come in contact or at least be physically hinder a bending direction that brings them closer to each other. This effect is of course less pronounced for nonwovens which have been deformed with protrusions on both sides of the nonwoven (e.g. SoS).

### EXAMPLES OF DEFORMED NONWOVEN ACCORDING TO THE INVENTION

In the following examples, all % are weight % unless indicated otherwise. The precursor nonwovens are indicated with their properties before deformation. Different alternative mechanical deformation treatments were conducted, and the properties of deformed nonwovens were measured as indicated below.

### Example 1: Bi-Layer Nonwoven Comprising a Carded Nonwoven and an Airlaid Layer

| Precursor NW Material | Bi-layer material with: | |
|---|---|---|
| | a 60 gsm airlaid nonwoven (3% latex, | |
| | 20% PP/PET bicopolymer, 77% | |
| | cellulose fibers), and | |
| | a 20 gsm PE/PP carded air-through | |
| | carrier nonwoven | |
| Precursor Horizontal | 14 | mm (for both sides) |
| Bending Drop value | | |
| Precursor Z-Compliance | 44.9 | mm³/N |
| Index | | |

| | |
|---|---|
| Precursor Recovery | 77.7% |
| Index | |

While this precursor has a relatively good Z-Compliance Index and Recovery Index, the precursor nonwoven has a very low Bending Drop Value and thus would make a diaper very stiff.

| Treatment A | Nested Self treatment (as in FIG. 22 of | |
|---|---|---|
| | WO 2016/040101) with a DOE 2.5 mm | |
| Horizontal Bending | 91 | mm (protrusions facing upwards) |
| Drop value after | 51 | mm (protrusions facing downwards) |
| Treatment A | | |
| Z-Compliance Index | 96.3 | mm³/N |
| after treatment A | | |
| Recovery Index After | 48.3% | |
| treatment A | | |
| Caliper at 0.85 kPa | 1.99 | mm |
| After Treatment A | | |

| Treatment B | Self-on-Self treatment (as in FIG. 16 and | |
|---|---|---|
| | following of WO2012/148,944A1) with a | |
| | DOE 3.0 mm | |
| Horizontal Bending | 91 | mm (Side 1 facing upwards) |
| Drop value after | 82 | mm (Side 2 facing downwards) |
| Treatment B | | |
| Z-Compliance Index | 100.3 | mm³/N |
| after treatment B | | |
| Recovery Index after | 53.1% | |
| treatment B | | |
| Caliper at 0.85 kPa | 2.15 | mm |
| after Treatment B | | |

Both mechanical deformation treatments increased the Bending Drop value dramatically as well as the Z-Compliance Index. The mechanical deformations broke the usual trade-off between flexibility and cushiness. The Recovery Index of the mechanically treated decreased but is still acceptable.

### Example 2: MBAL

| Precursor NW Material | 100 gsm MBAL airlaid nonwoven (5% Latex, | |
|---|---|---|
| | 19% PE/PET bicopolymer, 76% Cellulose) | |
| Precursor Horizontal | 17 | mm (for both sides) |
| Bending Drop value | | |
| Precursor Z-Compliance | 68.2 | mm³/N |
| Index | | |

| | |
|---|---|
| Precursor Recovery | 76.9% |
| Index | |

| Treatment A | Nested Self treatment (as in FIG. 22 of | |
|---|---|---|
| | WO 2016/040101) with a DOE 2.75 mm | |
| Horizontal Bending | 84 mm (protrusions facing upwards, other | |
| Drop value after | side was clearly lower but not measured) | |
| Treatment A | | |
| Z-Compliance Index | 124.1 | mm³/N |
| after Treatment A | | |
| Recovery index after | 50.8% | |
| Treatment A | | |
| Caliper at 0.85 kPa | 2.62 | mm |
| after Treatment A | | |

| Treatment B | Self-on-Self treatment (as in FIG. 16 and | |
|---|---|---|
| | following of WO2012/148,944A1) with a | |
| | DOE 3.0 mm | |
| Horizontal Bending | 90 | mm (Side 1 facing upwards) |
| Drop value after | 89 | mm (Side 2 facing downwards) |
| Treatment B | | |
| Z-Compliance Index | 110.3 | mm³/N |
| after Treatment B | | |
| Recovery index after | 60.7% | |
| Treatment B | | |
| Caliper at 0.85 kPa after | 2.65 | mm |
| Treatment B | | |

This shows the same effect as for the first example above for a different class of material (MBAL). The Depth of Engagement (DOE) is adapted to the different materials and treatments considered for the best effect.

| Treatment C | “Self” treatment using two rolls as on | |
|---|---|---|
| | FIG. 6 of US8,502,013 with a DOE 1.5 mm (but | |
| | configured to have deformed regions each | |
| | having 3 rib-like elements instead of the | |
| | 9 shown on FIG. 10 of US8,502,013) | |
| Horizontal Bending | 88 | mm (protrusions facing upwards) |
| Drop value after | 82 | mm (protrusions facing downwards) |
| Treatment C | | |
| Z-Compliance Index | 59.7 | mm3/N |
| after treatment C | | |
| Recovery index after | 73.9% | |
| Treatment C | | |
| Caliper at 0.85 kPa | 1.83 | mm |
| after Treatment C | | |

This treatment C was not as effective as the two previous treatments A and B in breaking the trade-off between flexibility and cushiness, but it still improved the Horizontal Bending Drop values. A lower DOE was used in treatment C because higher DOE caused material integrity issues for this treatment.

### Example 3: Spunlace

| Precursor NW Material | A 110 gsm integrated spunlace nonwoven | |
|---|---|---|
| | comprised of three carded strata having | |
| | the following composition: | |
| | Card 1 + Card 2 (37 gsm each): | |
| | 20% 1.7 | dtex CV |
| | 40% 5.8 | dtex PET/PE |
| | 40% 10 | dtex PET HS |
| | Card 3 (36 gsm): | |
| | 80% 1.7 | dtex CV |
| | 20% 10 | dtex PET HS |
| | Activated with a temperature above 130 C. | |
| Precursor Horizontal | 60 | mm (for both sides) |
| Bending Drop value | | |
| Precursor Z-Compliance | 36.6 | mm³/N |
| Index | | |

| | |
|---|---|
| Precursor Recovery | 79.5% |
| index | |

| Treatment A | Self-on-Self treatment (as in FIG. 16 and | |
|---|---|---|
| | following of WO2012/148,944A1) with a | |
| | DOE 4.6 mm | |
| Horizontal Bending Drop | 83 | mm (Side 1 facing upwards) |
| value after Treatment A | 86 | mm (Side 2 facing downwards) |
| Z-Compliance Index | 145.5 | mm³/N |
| after Treatment A | | |
| Recovery index after | 66.6% | |
| Treatment A | | |
| Caliper at 0.85 kPa | 3.21 | mm |
| after Treatment A | | |

| Treatment B | Self-on-Self treatment (as in FIG. 16 and | |
|---|---|---|
| | following of WO2012/148,944A1) with a | |
| | DOE 3.0 mm | |
| Horizontal Bending Drop | 90 | mm (Side 1 facing upwards) |
| value after Treatment B | 87 | mm (Side 2 facing downwards) |
| Z-Compliance Index | 77.5 | mm³/N |
| after treatment B | | |
| Recovery Index after | 73.5% | |
| Treatment B | | |
| Caliper at 0.85 kPa | 2.02 | mm |
| after Treatment B | | |

Treatments A and B show how the DOE can impact the desired flexibility and cushiness of the deformed nonwovens. Both properties were improved at the different DOE values, with higher DOE as in treatment A increasing the size of the protrusions and thus higher compliance, however the effect on the horizontal bending drop value is reduced compared to a similar treatment at a lower DOE as in treatment B. Both deformed nonwovens obtained by treatment A and treatment B are however generally satisfactory for use in the invention.

### General Description of an Absorbent Article 20

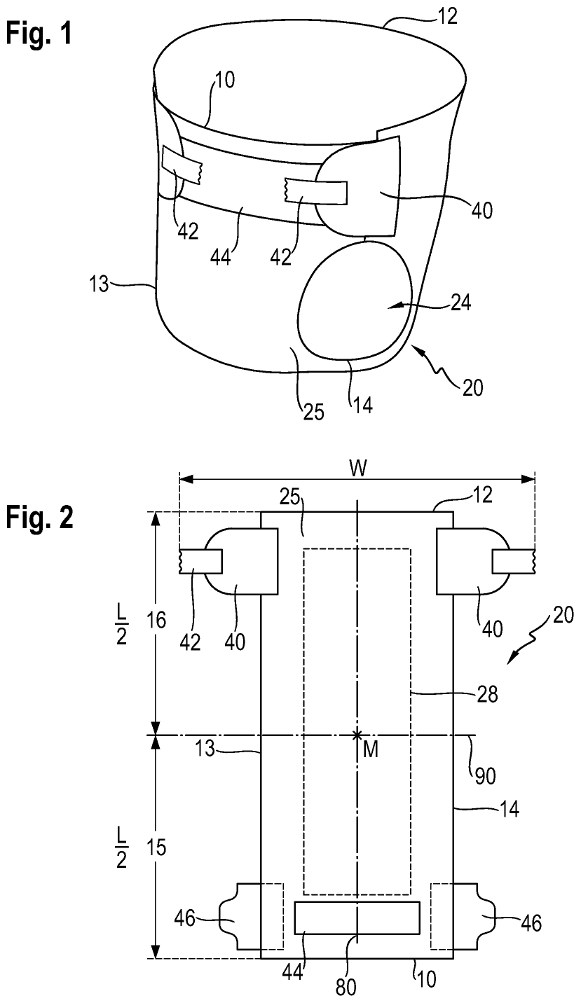

An exemplary absorbent article that may use a nonwoven of the invention is represented in FIGS. 1-4 in the form of a baby taped diaper 20. This taped diaper 20 is shown for illustration purpose only as the invention is applicable to a wide variety of diapers or other absorbent articles such as baby diaper pants, adult incontinence pants or feminine sanitary pads. In the following, the word “diaper” and “absorbent article” are used interchangeably. The Figures are used herein as illustration of one way to carry out the invention and are not limiting the scope of the claims, unless specifically indicated to do so.

The absorbent article 20 comprises a liquid permeable topsheet 24 on its wearer-facing surface, a liquid impermeable backsheet 25 on its garment-facing surface and an absorbent core 28 between the topsheet and the backsheet. The topsheet typically forms the majority of the wearer-contacting surface of the article and is the first layer that the body exudates contact. The topsheet is liquid permeable, permitting liquids to readily penetrate through its thickness. Any known topsheet may be used in the present invention. The backsheet typically comprises a fluid impermeable plastic film, which may be printed with a backsheet pattern, and a low basis weight nonwoven cover glued to this impermeable film to give a nicer feel and appearance to the backsheet.

The absorbent article has a longitudinal axis 80 extending longitudinally from the front edge 10 to the back edge 12 of the article and notionally dividing the article into a left and right half. The front and back edges 10, 12 form the waist opening of the diaper when put on the wearer. The articles also have a left and right longitudinal edges 13, 14 that form leg opening when the article is worn by the wearer. The length L of the article may be measured along the longitudinal axis 80. The absorbent article can also be notionally divided by a transversal axis 90 at half the length L. These axes are meeting at the center of the article M. The article can be further notionally divided in three regions having equal length of a third of L along the longitudinal axis: a front region extending from the front edge towards the crotch region for a third of L, a crotch region in the middle third of the diaper, and a back region extending from the crotch region to the back edge of the article for the remaining third of L. All three regions are of equal length measured on the longitudinal axis, when the article is in such a flat state. The front region, crotch region, back region and longitudinal and transversal axis are defined herein notionally, that is they are typically not materialized in the real diapers, but are useful to describe the positions of various components of the invention relative to each other and the diaper. The article also a width W, which is the maximum extension of the article as measured perpendicular to its length.

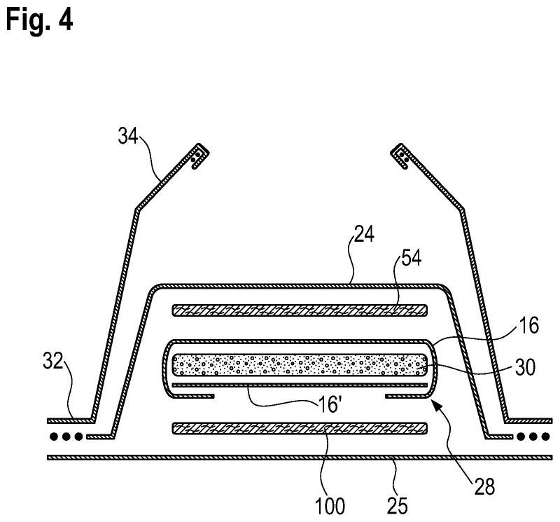

The absorbent article typically comprises a fluid acquisition layer 54 between the topsheet and the absorbent core. The fluid acquisition layer may be advantageously a deformed nonwoven as previously disclosed. Other typical diaper components include elasticized gasketing cuffs 32 and upstanding barrier leg cuffs 34, which are present in most diapers. The absorbent article may also comprise other known diaper components, which are not represented in the Figures, such as transverse barrier cuffs, front and/or back elastic waistbands, a lotion application on the topsheet, longitudinally extending channels in the core and/or the distribution layer, a wetness indicator, etc. . . . all these components have been described and exemplified in the art and are not further detailed herein. More detailed disclosures of example of such components are for example disclosed in WO201493323, WO2015/183669 (both Bianchi et al), WO 2015/031225 (Roe et al.) or WO2016/133712 (Ehrnsperger et al.) to name a few. The absorbent article of the invention may also comprise a masking layer 100, which may or may not be a deformed nonwoven as described previously. The masking layer may be a discrete layer disposed between the absorbent core and the backsheet, as illustrated in FIG. 4, or it may also be an integral part of the absorbent core such as a discrete layer disposed between the absorbent layer 30 and the bottom side of the core wrap 16. The masking layer may also be used to form the bottom side of the core wrap 16′, thus simplifying the construction of the diaper. Desirable properties for such masking layer are discussed further below.

The topsheet 24, the backsheet 25, the absorbent core 28, the nonwoven of the invention 60 and other article components may be assembled in a variety of well-known configurations, in particular by gluing, fusion and/or pressure bonding. The invention thus also encompasses a process for making an absorbent article comprising the steps of combining a nonwoven according to the present invention with the other absorbent article components, e.g. topsheet, backsheet and absorbent core.

### General Description of an Absorbent Core 28

The absorbent core 28 is the component of the absorbent article having the most absorbent capacity and comprises an absorbent material layer 30. The absorbent material layer 30 may be generally rectangular or shaped, for example sand-hour shaped with a tapering along its width towards the middle region of the core (when seen from the top). In this way, the absorbent material deposition area may have a relatively narrow width in an area of the core intended to be placed in the crotch region of the absorbent article. This may provide for example better wearing comfort. This is of course not limiting the scope of the invention as the invention is applicable to a wide variety of absorbent cores. Other shapes can also be used such as rectangular, a “T” or “Y” or “dog-bone” shape for the area of the absorbent material.

As illustrated in FIG. 4, the absorbent core typically comprises an absorbent layer 30 in a core wrap 16, 16′. The core wrap may be formed by a single layer or by two separate layers forming respectively the top side 16 and the bottom side 16′ of the absorbent core, and are attached to each at least longitudinally, for example as a C-wrap as illustrated in FIG. 4 (the attachment such as glue bonding are not represented for simplicity).

The absorbent material 30 may be any conventional absorbent material known in the art. For example, the absorbent material may comprise a blend of cellulose fibers and superabsorbent particles (“SAP”), typically with the percentage of SAP ranging from 40% to 70% by weight of the absorbent material. The absorbent material layer 30 may advantageously also be free of cellulose fibers, as is known in so-called airfelt-free cores where the absorbent material consists of SAP. Airfelt-free cores are typically much thinner compared to conventional cellulose fibers comprising cores, and may thus be particularly useful when combined with an acquisition layer made of a nonwoven according to the present invention.

“Superabsorbent polymers” or “SAP” as used herein refer to absorbent material which are cross-linked polymeric materials that can absorb at least 10 times, preferably at least 15 times, their weight of an aqueous 0.9% saline solution as measured using the Centrifuge Retention Capacity (CRC) test (EDANA method WSP 241.2-05E). These polymers are typically used in particulate forms so as to be flowable in the dry state. The term “particles” refers to granules, fibers, flakes, spheres, powders, platelets and other shapes and forms known to persons skilled in the art of superabsorbent polymer particles.

Various absorbent core designs comprising high amount of SAP have been proposed in the past, see for example in U.S. Pat. No. 5,599,335 (Goldman), EP1,447,066 (Busam), WO95/11652 (Tanzer), US2008/0312622A1 (Hundorf), WO2012/052172 (Van Malderen). In particular the SAP printing technology as disclosed in US2006/024433 (Blessing), US2008/0312617 and US2010/0051166A1 (both to Hundorf et al.) may be used. The invention is however not limited to a particular type of absorbent core. The absorbent core may also comprise one or more glue such as auxiliary glue applied between the internal surface of one (or both) of the core wrap layers and the absorbent material to reduce leakage of SAP outside the core wrap. A micro-fibrous adhesive net may also be used in air-felt free cores, as described in the above Hundorf references. These glues are not represented in the Figures for simplicity.

The absorbent material may be for example deposited as a continuous layer between the top layer 16 and the bottom layer 16′ of the core wrap. The core wrap is typically comprised of a low basis weight nonwoven, e.g. a SMS material (spunbond-meltblown-spunbond laminate). The absorbent material may also be present discontinuously for example as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern wherein the resulting layer is substantially continuously distributed across the absorbent particulate polymer material area. As for example taught in US2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

Alternatively, the absorbent article may comprise an absorbent core comprising a fluid-permeable top layer, a bottom layer and a central layer between the top layer and the bottom layer, wherein the central layer is or comprises a high loft fibrous nonwoven layer in which the superabsorbent particles are provided within the pores of the nonwoven layer. The high loft nonwoven can be for example a carded web comprising synthetic fibers having a basis weight of from 10 gsm to 70 gsm. Examples of such cellulose fiber free cores are disclosed in WO2016106021A1 (Bianchi et al., P&G).

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core.

The absorbent core may also comprise one or more longitudinally extending channels (not represented) which are areas substantially free of absorbent material within the absorbent material layer. The core wrap may be bonded through these material-free areas. Exemplary disclosures of such channels in an airfelt-free core can be found in WO2012/170778 (Rosati et al.) and US2012/0312491 (Jackels et al.). Each channel may e.g. have a length as measured in the MD which is at least 10% of the length L of the article, in particular from 15% to 80% of the length L of the article. Channels may of course also be formed in absorbent cores comprising cellulose fibers. Channels can improve the flexibility of the articles, in particular in the CD direction, as well as the speed of acquisition of the speed in the core as they can transport fluid quickly towards the front and back of the ocre.

### Acquisition Layer 54

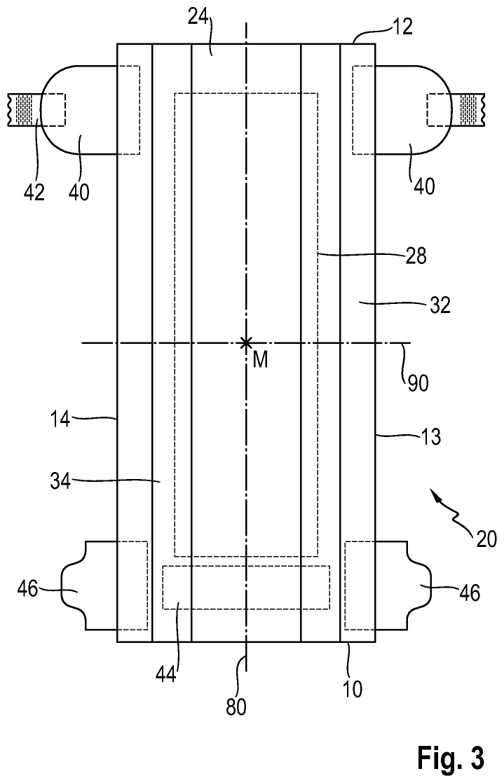

As indicated previously, the deformed nonwoven of the present information can be advantageously used as an acquisition layer 54 disposed between the topsheet 24 and the absorbent core 28. The acquisition layer 54 may have any suitable size, and may be smaller, larger or same size as the absorbent material layer 30 or the absorbent core 28 (as considered when the diaper is flattened out as in FIG. 3). The acquisition layer is typically hydrophilic, as defined by a contact angle with deionized water at 22° C. of less than 90°, typically a contact angle of less than 70°. The contact angle may be more easily measured on the precursor nonwoven.

The protrusions of the deformed nonwoven may be advantageously oriented towards the absorbent core so that they do not “push” into the topsheet. However, they may also be oriented towards the topsheet if desired. Some deformed nonwovens described above also have protrusions on both sides of the nonwovens (see SoS process described above) so that some protrusions may also extend towards the topsheet and the other towards the absorbent core.

The absorbent article may also comprise an additional acquisition layer (not represented), in particular between an acquisition layer 54 that is a nonwoven according to the invention and the topsheet. Such an additional acquisition layer may be particularly useful if the deformed nonwoven is not already a multi-layer nonwoven having sub-layers (as illustrated in FIGS. 6, 8 and 10) with different fluid acquisition and distribution properties. Such additional acquisition layer may in particular be an air-through bonded carded nonwoven, for example having a basis weight of from 20 gsm to 100 gsm, alternatively from 30 gsm to 80 gsm. Such additional acquisition layer is typically made of synthetic fibers that have been hydrophillically treated with a surfactant, as is known in the art. The first and second acquisition layers may form together an acquisition system. The deformed nonwoven according to the invention may alternatively be integrated in the absorbent core, such as forming the top side of the core wrap or being disposed between a top side 16 of the core wrap and the absorbent layer 30.

### Masking Layer 100

The absorbent article may optionally comprise a masking layer 100 disposed between the absorbent core 28 and the backsheet 25, or more generally between the absorbent layer 30 and the backsheet 25 if it is integrated with the absorbent core 28. The masking layer may help improving the feel of the absorbent article as perceived from the garment-facing side by masking the potentially gritty feel of the superabsorbent particles in the absorbent core. The masking layer may be provided by a nonwoven material, a film, a foam, or other suitable material, but in particular a deformed nonwoven according to the invention. In this latter case, the masking layer may be the sole deformed nonwoven according to the invention in the article, or may be used in combination with a deformed nonwoven as acquisition layer, as discussed above. The masking layer is typically hydrophobic, so as to provided a barrier between the absorbent layer and the backsheet. The masking layer may thus have a contact angle with deionized water at 22° C. of more than 90°, typically a contact angle of more than 100°. The contact angle may be more easily measured on the precursor nonwoven if the masking layer is mechanically deformed.

The masking layer may typically be a discrete layer 100 disposed between the absorbent core and the backsheet, as illustrated in FIG. 4, alternatively the masking layer may also be an integral part of the absorbent core such as a discrete layer disposed between the absorbent layer 30 and the bottom side of the core wrap 16′. The masking layer may also be used to form the bottom side of the core wrap 16′, thus simplifying the construction of the diaper.

Typically, the thickness of the masking layer should be large enough to mask any gritty feeling of superabsorbent polymers in the absorbent core, while having a low enough of a stiffness to allow the absorbent article to remain flexible and conform to a wearer. A thickness (C1) of from 0.2 mm to 4.00 mm, preferably of from 0.35 mm to 2.00 mm, as measured at a pressure of 0.85 kPa according to the Z-Compliance Index and Percent Recovery Measurement Method described herein, and a basis weight ranging of from about 20 grams per square meter (gsm) to about 100 gsm, preferably from 30 gsm to 75 gsm, whilst not limiting the present disclosure, may be advantageous.

The masking layer, even if it is not a deformed nonwoven as described above, advantageously has a Horizontal Bending Drop at 100 mm of at least 60 mm. Of course, higher values are advantageous, and can be obtained by deforming a nonwoven as disclosed above. The masking layer may thus also have Horizontal Bending Drop of at least 75 mm, preferably at least 80 mm, and more preferably at least 85 mm, as measured with the Horizontal Bending Drop at 100 mm Measurement Method described herein.

The masking layer may also have advantageously have a Z-Compliance Index of at least 10 mm³/N, preferably at least 15 mm³/N, as measured according to the Z-Compliance Index and Percent Recovery Measurement Method described herein. Higher values such as those for the mechanically deformed nonwovens are of course advantageous, the masking thus can have a Z-Compliance Index of at least 50 mm³/N, or even at least 60 mm³/N, as measured according to the Z-Compliance Index and Percent Recovery Measurement Method described herein.

### Examples of Masking Layer 100

The following examples have not been mechanically deformed, as the masking layer if used in the invention may or may or not be mechanically deformed.

| Example 1 | A 60 gsm Carded air through bonded | |
|---|---|---|
| | nonwoven made of 100% polyethylene/PET | |
| | solid round bicomponent (core/sheath) | |
| | staple fibers of 4.4 dtex. | |
| Example 1 Horizontal | 51 | mm (side 1) |
| Bending Drop value | 66 | mm (side 2) |
| Example 1 Z-Compliance | 31.8 | mm³/N |
| Index | | |
| Example 1 Caliper at | 0.79 | mm |
| 0.85 kPa | | |

| Example 2 | Bilayer Carded Calendar bonded (hydrophobic) | |
|---|---|---|
| | Card 1: 30 g/m² - 70% PET 6.7 dtex | |
| | 30% PP 2.2 dtex | |
| | Card 2: 10 g/m² -100% PP | |
| Example 2 Horizontal | 90 | mm (Side 1) |
| Bending Drop value | 92 | mm (Side 2) |
| Example 2 Z-Compliance | 30.0 | mm³/N |
| Index | | |
| Example 2 Caliper at | 0.67 | mm |
| 0.85 kPa | | |

| Example 3 | 20 gsm SMS 100% PP Nonwoven | |
|---|---|---|
| | S layer: 9.1 gsm Spunbond (each) with | |
| | fibers of 2.2 dtex | |
| | M layer: 1.8 gsm meltblown layer with | |
| | fiber diameter lower than 2 microns. | |
| Example 3 Horizontal | 91 | mm (Side 1) |
| Bending Drop value | 93 | mm (Side 2) |
| Example 3 Z-Compliance | 4.3 | mm³/N |
| Index | | |
| Example 3 Caliper at | 0.18 | mm |
| 0.85 kPa | | |

This non-mechanically deformed example 3 has a good drapability (>90 mm) and can be for example used as bottom core wrap layer, however its compliance index is relatively low to provide an effective masking effect and therefore is not.

### Packages

Absorbent articles for personal hygiene are typically packaged by the manufacturer in a plastic bag and/or a cardboard box for transport and sale. The articles may also be folded before being packaged to save space as is known in the art. The back and front ears of taped diapers are for example typically folded inwardly before bi-folding the diaper along its transversal axis before being packaged. The absorbent articles may be packaged under compression, so as to reduce the size of the package so that the caregivers can easily handle and store the packages, while also providing distribution and inventory savings to manufacturers owing to the size of the packages. Care should still be taken not to compress too much the protrusions so that they keep their shape during storage and transport or at least can recover after being extracted from the packages. Typical packages comprise amount of articles ranging from 2 to 200 of the articles.

Packages of the absorbent articles of the present disclosure may in particular have an In-Bag Stack Height of less than 120 mm, or less than 110 mm, or less than 105 mm, or less than 100 mm, or less than 95 mm, or less than 90 mm, as measured according to the In-Bag Stack Height Test described herein. For each of the values indicated in the previous sentence, it may be desirable to have an In-Bag Stack Height of greater than 60 mm, or greater than 70 mm, or greater than 75 mm, or greater than 80 mm. Packages of the absorbent articles of the present disclosure may thus have an In-Bag Stack Height of from 60 mm to 120 mm, or from 75 mm to 110 mm, or from 80 mm to 110 mm, or from 80 mm to 105 mm, or from 80 mm to 100 mm, as measured according to the In-Back Stack Height Test described herein.

### Test Methods

The values indicated herein are measured according to the methods indicated herein below, unless specified otherwise. All measurements are performed at 23° C.±2° C. and 50%±2% RH, unless specified otherwise. All samples should be kept at least 24 hours in these conditions to equilibrate before conducting the tests, unless indicated otherwise. If possible, measurements are made on the component materials before they are integrated in an absorbent article. If this is not possible, care should be exerted when excising the sample to not impart any contamination or distortion to the test sample layer during the removal the material from other layers (using cryogenic spray, such as Cyto-Freeze, Control Company, Houston, Tex., if needed).

### Horizontal Bending Drop at 100 mm (HBD@100 mm) Measurement Method

Principle: this method measures the ability of a nonwoven to bend under his own weight (sometimes designated as “drapability”). The measurement principle is to hang a length of 100 mm of the material over a sharp 90° edge and measure the vertical drop of this length of the material under its own weight, expressed in mm. This vertical drop is illustrated as reference number 1 in FIG. 11.

Apparatus: the setup for conducting the measurement is schematically shown in FIG. 1 and comprises:
i) a flat support box 2 made of any suitable material such as polycarbonate (e.g. Lexan®) about 400 mm long, about 200 mm wide and a height 3 of exactly 140 mm, with at least one of its top edge 4 in the width direction having a sharp 90 degree angle. The box 1 is positioned on a suitable flat surface 5, such as a lab bench;
ii) a movable vertical metal ruler 6, having a stable horizontal foot, and calibrated so that its zero corresponds to the flat surface 5 on which the box is disposed. The movable vertical metal ruler is used to measure the distance 7 of the hanging edge 8 of the material specimen 9 to the flat surface.

Procedure: a rectangular material specimen 9 having a width of about 80 mm and a length of about 200 mm is cut from a roll stock of the nonwoven. The length corresponds to the machine direction of the nonwoven and the width corresponds to the cross-direction of the nonwoven. The method can be alternatively conducted on a material specimen having a width of about 50 mm if the nonwoven original's width is shorter than 80 mm.

The material specimen 9 is laid flat on any suitable horizontal flat surface such as a lab bench, and a line is drawn at exactly at 100 mm from the front edge 8 of the material specimen in the width direction.

The material specimen 9 is then laid on top of the support box 2 with a first side of the specimen facing up (side A). The 100 mm line drawn is precisely positioned on the sharp edge 4 with the 100 mm long portion of the material specimen hanging free from the support box 2, as illustrated on FIG. 11. The section of the sample is held flush on the horizontal side of the sharp edge if needed.

The movable ruler 6 is positioned near the front edge 8 of the hanging specimen material so that the distance 7 of the hanging front edge 8 from the flat surface 5 can be measured. Since the hanging front edge 8 may not be perfectly horizontal, the distance is measured on the two corners of the hanging front edge 8, as well as in the center of the front edge 8, and the arithmetic mean of the three values recorded to the nearest mm.

The bending drop 1 is calculated as the difference between the exact Drape box height 3 (140 mm) and the recorded vertical distance 7 of the front edge 8 to the flat surface 5, as measured with the ruler 6 from the flat surface 5.

The material specimen is then turned upside down (side B now up), and the same procedure described above is performed.

The overall procedure above is repeated on five like material specimens. The arithmetic mean of the bending drop values for the five like material specimens is reported to the nearest mm as the Horizontal Bending Drop at 100 mm (HBD@100 mm) for each side of the nonwoven tested. The bending drop recorded overall for the material specimen is the greater of the side A mean bending drop value and the side B mean bending drop value.

### Z-Compliance Index and Percent Recovery Measurement Method

Principle: this method measures the ability of a nonwoven to be compressed in z-direction under applied pressure and then to recover to its original caliper after removing the applied pressure.

Setup: a vertically oriented electronic caliper tester having a precision of at least 0.01 mm with a 40 mm diameter circular foot may be used. The pressure exerted by the foot on the specimen is adjustable via the addition of pre-selected weights. Measurements are made at 0.85±0.05 kPa and 15.4±0.1 kPa.

Procedure: A material specimen is cut from the nonwoven roll stock to a square sample with a width of about 80 mm (or alternatively in case the material is not available in the suitable size in a material specimen with a width of about 50 mm).

The square sample specimen is positioned centered under the caliper foot and the caliper at 0.85±0.05 kPa (P1) is measured and recorded to the nearest 0.01 mm (C1). Without removing the sample from the equipment, the pressure is increased to 15.4±0.1 kPa (P2) and the caliper measured and recorded to the nearest 0.01 mm (C2). The pressure may be increased by adding a suitable weight on the caliper foot. Again without moving the sample, the exerted pressure is reduced back to 0.85±0.05 kPa (for example by removing the extra weight) and the caliper measured a third time (C3) and recorded to the nearest 0.01 mm.

For the specimen being measured, the compliance index is defined as:

Z-compliance index=(C1−C2)/(P2−P1)

and is recorded to the nearest 0.1 mm³/N.

The recovery is calculated as:

recovery=C3/C1*100%

expressed in percent and recorded to the nearest 0.1%.

The procedure above is conducted on five like specimens of the same nonwoven. The arithmetic mean of the compliance index values among the five specimens is calculated and reported to the nearest 0.1 mm³/N as the Compliance Index. The arithmetic mean of percent recovery values among the five specimens is calculated and reported to the nearest 0.1% as the Percent Recovery.

### In-Bag Stack Height Test

The In-Bag Stack Height (IBSH) of a package of absorbent articles is determined as follows:

Equipment: A thickness tester with a flat, rigid horizontal sliding plate is used. The thickness tester is configured so that the horizontal sliding plate moves freely in a vertical direction with the horizontal sliding plate always maintained in a horizontal orientation directly above a flat, rigid horizontal base plate. The thickness tester includes a suitable device for measuring the gap between the horizontal sliding plate and the horizontal base plate to within ±0.5 mm. The horizontal sliding plate and the horizontal base plate are larger than the surface of the absorbent article package that contacts each plate, i.e. each plate extends past the contact surface of the absorbent article package in all directions. The horizontal sliding plate exerts a downward force of 850±1 gram-force (8.34 N) on the absorbent article package, which may be achieved by placing a suitable weight on the center of the non-package-contacting top surface of the horizontal sliding plate so that the total mass of the sliding plate plus added weight is 850±1 grams.

Test Procedure: Absorbent article packages are equilibrated at 23±2° C. and 50±5% relative humidity prior to measurement. The horizontal sliding plate is raised and an absorbent article package is placed centrally under the horizontal sliding plate in such a way that the absorbent articles within the package are in a horizontal orientation. Any handle or other packaging feature on the surfaces of the package that would contact either of the plates is folded flat against the surface of the package so as to minimize their impact on the measurement. The horizontal sliding plate is lowered slowly until it contacts the top surface of the package and then released. The gap between the horizontal plates is measured to within ±0.5 mm ten seconds after releasing the horizontal sliding plate. Five identical packages (same size packages and same absorbent articles counts) are measured and the arithmetic mean is reported as the package width. The “In-Bag Stack Height”=(package width/absorbent article count per stack)×10 is calculated and reported to within ±0.5 mm.

### Misc

As used herein, the terms “comprise(s)” and “comprising” are open-ended; each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essentially of” which excludes any element, step or ingredient not mentioned which materially affect the way the feature performs its function, and the term “consisting of” which excludes any element, step, or ingredient not specified. Any preferred or exemplary embodiments described below are not limiting the scope of the claims, unless specifically indicated to do so. The words “typically”, “normally”, “preferably”, “advantageously”, “in particular” and the likes also qualify features which are not intended to limit the scope of the claims unless specifically indicated to do so.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A mechanically deformed nonwoven having a first surface and a second surface, the nonwoven comprising a plurality of protrusions extending outwardly at least from the first surface and having openings corresponding to the protrusions on the second surface, characterized in that the nonwoven has:
a Horizontal Bending Drop at 100 mm of at least 75 mm, as measured with the Horizontal Bending Drop at 100 mm Measurement Method described herein; and
a Z-Compliance Index of at least 50 mm³/N, as measured according to the Z-Compliance Index and Percent Recovery Measurement Method described herein.

2. The mechanically deformed nonwoven according to claim 1, wherein the protrusions comprise a base proximate the first surface of the nonwoven, an opposed distal end extending outward in the Z-direction from the base, side walls between the base and the distal end of the protrusion, and a cap comprising at least a portion of the side walls and the distal end of the protrusions, wherein the side walls have interior surfaces, wherein the interior surfaces of the side walls define a base opening at the base of the protrusion, wherein the cap has a portion with a maximum interior width (Wi), and the base opening has a width (Wo), and wherein the maximum interior width of the cap of the protrusion is greater than the width of the base opening.

3. The mechanically deformed nonwoven according to claim 1, wherein at least some of the protrusions have a secondary opening at their cap.

4. The mechanically deformed nonwoven according to claim 1, wherein the nonwoven further comprises protrusions extending outwardly from the second surface of the nonwoven and having openings corresponding to these opposite protrusions on the first surface of the nonwoven.

5. The mechanically deformed nonwoven according to claim 1, wherein the nonwoven has a thickness of from about 0.50 mm to about 4.00 mm, as measured at a pressure of 0.85 kPa according to the Z-Compliance Index and Percent Recovery Measurement Method described herein.

6. The mechanically deformed nonwoven according to claim 1, wherein the nonwoven has a basis weight ranging of from about 20 grams per square meter (gsm) to about 200 gsm.

7. The mechanically deformed nonwoven according to claim 1, wherein the nonwoven was obtained by deforming a precursor nonwoven which is selected from airlaids, composite nonwovens or integrated nonwovens.

8. The mechanically deformed nonwoven according to claim 7, wherein the precursor nonwoven is an integrated nonwoven, in particular a spunlace comprising at least one strata having a plurality of absorbent fibers, a plurality of stiffening fibers and a plurality of resilient fibers.

9. The mechanically deformed nonwoven according to claim 1, wherein the nonwoven has a Percent Recovery of at least 30%, as measured by the Z-Compliance Index and Percent Recovery Measurement Method described herein.

10. An absorbent article comprising a topsheet, a backsheet, an absorbent core, and at least one mechanically deformed nonwoven according to claim 1, wherein the absorbent core comprises an absorbent layer and a core wrap, and wherein the absorbent layer comprises superabsorbent particles and is optionally free of cellulose fibers.

11. The absorbent article according to claim 10, wherein the mechanically deformed nonwoven is:
an acquisition layer between the topsheet and the absorbent core; and/or
a masking layer between the absorbent layer and the backsheet.

12. The absorbent article according to claim 11, wherein the mechanically deformed nonwoven is an acquisition layer disposed between the absorbent core and the topsheet, and wherein the absorbent article further comprises an additional acquisition layer between the topsheet and the mechanically deformed nonwoven.

13. The absorbent article according to claim 10, wherein the article comprises a masking layer, the masking layer having a Horizontal Bending Drop at 100 mm of at least 60 mm and/or a Z-Compliance Index of at least 10 mm³/N.

14. The absorbent article according to claim 13, wherein the masking layer is a mechanically deformed nonwoven according to claim 1.

15. The absorbent article according to claim 10, wherein the protrusions extending outwardly from the first surface of the mechanically deformed nonwoven are oriented towards the absorbent core of the absorbent article.

16. A package comprising a plurality of absorbent articles according to claim 10, in particular wherein the package has an In-Bag Stack Height ranging from about 60 mm to about 120 mm, as measured according to the In-Bag Stack Height Test described herein.
